(19) United States
(12) Patent Application Publication
(10) Pub. No.: US 2025/0076427 A1
(43) Pub. Date: Mar. 6, 2025

(54) Wireless Transmission Device and Magnetic Resonance Imaging Apparatus

(71) Applicant: Siemens Healthineers AG, Forchheim (DE)

(72) Inventors: JianMin Wang, Shenzhen (CN); Markus Vester, Nürnberg (DE); Jan Bollenbeck, Eggolsheim (DE)

(73) Assignee: Siemens Healthineers AG, Forchheim (DE)

(21) Appl. No.: 18/817,886

(22) Filed: Aug. 28, 2024

(30) Foreign Application Priority Data
Aug. 31, 2023 (CN) 202311128245.8

(51) Int. Cl. G01R 33/36

(52) U.S. Cl. CPC G01R 33/3692

(57) ABSTRACT

A wireless transmission device for an MR imaging apparatus, including: a transmitting coupler with a transmitting coil array including one or more first transmitting coils arranged in a first local coil device and to transmit a first intermediate frequency signal, the first local coil device being located on a patient couch and below an examined portion of a subject, wherein the first intermediate frequency signal is generated based on a first magnetic resonance signal received by the first local coil device from the subject; and a first receiving coupler including a first receiving coil array, the first receiving coil array including one or more first receiving coils, located below a trajectory of the patient couch sliding over the magnetic resonance main body, and configured to receive the first intermediate frequency signal from the transmitting coil array in response to the transmitting coil array overlapping with the first receiving coil array.

### TECHNICAL FIELD

The present disclosure relates to the technical field of medical instruments, and in particular to a wireless transmission device and a magnetic resonance imaging apparatus.

### BACKGROUND ART

In a magnetic resonance imaging (MRI) apparatus, a local coil for receiving a magnetic resonance signal emitted by a subject is generally connected to a magnetic resonance control terminal via a cable and a connector. That is, the magnetic resonance signal received by the local coil is transmitted to the control terminal through the cable. However, signal transmission through cables has at least the following defects: 1) traps (e.g., traps located below a patient couch) used are usually very expensive; and 2) connectors (e.g., slots)used are usually difficult to clean. Therefore, it is desirable to provide wireless transmission between the local coil and the magnetic resonance control terminal.

The method described in this section is not necessarily a previously envisaged or used method. Unless otherwise stated, it should not be assumed that any method described in this section is regarded as prior art simply because it is included in this section. Similarly, unless otherwise stated, problems mentioned in this section should not be regarded as having been generally acknowledged in any prior art.

### SUMMARY

In view of this, an aspect of the present disclosure proposes a wireless transmission device for a magnetic resonance imaging apparatus. The wireless transmission device includes: a transmitting coupler including a transmitting coil array, the transmitting coil array including one or more first transmitting coils arranged in a first local coil device and being configured to transmit a first intermediate frequency (IF) signal, the first local coil device being located on a patient couch and below an examined portion of a subject, wherein the first IF signal is generated based on a first magnetic resonance signal received by the first local coil device from the subject; and a first receiving coupler including a first receiving coil array, the first receiving coil array including one or more first receiving coils and being located below a trajectory of the patient couch sliding over the magnetic resonance main body, and the first receiving coil array being configured to receive the first IF signal from the transmitting coil array in response to the transmitting coil array at least partially overlapping with the first receiving coil array.

Another aspect of the present disclosure provides a magnetic resonance imaging apparatus, including: a magnetic resonance main body; a patient couch; a wireless transmission device as described in the present disclosure; and a first local coil device, the first local coil device further including: a first magnetic resonance coil configured to detect a first magnetic resonance signal emitted by a subject; and a first frequency converter configured to convert the first magnetic resonance signal into a first IF signal.

It should be understood that the content described in this section is not intended to identify key or important features of aspects of the present disclosure, and not intended to limit the scope of the present disclosure. Other features of the present disclosure will become easy to understand through the following description.

### BRIEF DESCRIPTION OF THE DRAWINGS

The above-mentioned and other features and advantages of the present invention will become more apparent to those skilled in the art from the following detailed description of aspects of the present invention with reference to the accompanying drawings, in which

FIG. 1 shows a structural diagram of a magnetic resonance imaging apparatus according to an aspect of the present disclosure;

FIG. 2 shows a structural diagram of a first receiving coupler of a wireless transmission device according to an aspect of the present disclosure;

FIG. 3 shows a structural diagram of a transmitting coupler of the wireless transmission device according to the aspect of the present disclosure;

FIGS. 4A-4B show schematic diagrams of wireless transmission between the first receiving coupler and the transmitting coupler according to the aspect of the present disclosure;

FIG. 5 shows a schematic diagram of transmission of a first IF signal in the wireless transmission device according to the aspect of the present disclosure;

FIG. 6 shows a structural diagram of a main body-side transmission component of the wireless transmission device according to the aspect of the present disclosure;

FIG. 7 shows a structural diagram of a second receiving coupler of the wireless transmission device according to the aspect of the present disclosure;

FIG. 8 shows a structural diagram of a local coupler of the wireless transmission device according to the aspect of the present disclosure;

FIG. 9 shows a schematic diagram of transmission of a second IF signal in the wireless transmission device according to the aspect of the present disclosure; and

FIGS. 10A-10C show schematic diagrams of application scenarios of operating a magnetic resonance imaging apparatus according to an aspect of the present disclosure.

Reference signs in the accompanying drawings are as follows:
10 Magnetic resonance main body
11 Field magnet
12 Gradient coil
13 Body coil
14 Magnetic resonance cavity
20 Patient couch
21 Local coupler
21a Local coupling coil array
21b Coupling auxiliary coil
30 Main body-side transmission component
31 First receiving coupler
31a First receiving coil array
31b First receiving auxiliary coil
32 Second receiving coupler
32a Second receiving coil array
32b Second receiving auxiliary coil
40 First local coil device
41 Transmitting coupler
41a Transmitting coil array
41b Transmitting auxiliary coil
42 First frequency converter
43 First magnetic resonance coil
50 Second local coil device
51 Second frequency converter
52 Second magnetic resonance coil
60 Subject
70 Control terminal
80 Head coil device
81a Head transmitting coil array
81b Head transmitting auxiliary coil
90 Spine coil device
91a Spine transmitting coil array
91b Spine transmitting auxiliary coil

### Detailed Description

To enable a clearer understanding of the technical features, objectives, and effects of the present disclosure, particular aspects of the present disclosure are now explained with reference to the accompanying drawings, in which identical labels indicate identical parts.

As used herein, “schematic” means “serving as an instance, example, or illustration.” No drawing or aspect described herein as “schematic” should be interpreted as being a more preferred or more advantageous technical solution.

To make the drawings appear uncluttered, only those parts relevant to the present disclosure are shown schematically in the drawings; they do not represent the actual structure thereof as a product. Furthermore, to make the drawings appear uncluttered for ease of understanding, in the case of components having the same structure or function in certain drawings, only one of these is drawn schematically, or only one is marked.

In this text, “a” does not only mean “just this one”; it may also mean “more than one”. As used herein, “first” and “second,” etc., are merely used to differentiate between parts, not to indicate their order or degree of importance, or any precondition of mutual existence, etc.

In a magnetic resonance imaging apparatus, since a magnetic resonance signal emitted by a subject (e.g., a patient who needs to undergo a magnetic resonance examination) is very weak, it is usually necessary to arrange a receiving antenna (i.e., a local coil) as close as possible to an examined portion of the subject, such as the head, neck, spine, upper leg, lower leg, knee, ankle, foot, etc. In general, in order to transmit the magnetic resonance signal received by the local coil close to the subject to a control terminal of the magnetic resonance imaging apparatus for further processing (e.g., generating a magnetic resonance image), it is necessary to connect the local coil to the control terminal of the magnetic resonance imaging apparatus via a cable and a connector. For example, the local coil is connected to a slot on a patient couch, and the slot on the patient couch is then connected to a control terminal of a resonance system via a cable. However, a radio frequency trap used in the cable is usually very expensive, and the slot on the patient couch is also difficult to clean.

To solve the above problems, the present disclosure provides a wireless transmission device for a magnetic resonance imaging apparatus, and a magnetic resonance imaging apparatus.

According to an aspect of the present disclosure, the magnetic resonance imaging apparatus includes: a magnetic resonance main body; a patient couch; a wireless transmission device as described in the present disclosure; and a first local coil device, the first local coil device including: a first magnetic resonance coil configured to detect a first magnetic resonance signal emitted by a subject; and a first frequency converter configured to convert the first magnetic resonance signal into a first intermediate frequency signal.

According to an aspect of the present disclosure, the wireless transmission device includes: a transmitting coupler including a transmitting coil array, the transmitting coil array including one or more first transmitting coils arranged in a first local coil device and being configured to transmit a first IF signal, the first local coil device being located on a patient couch and below an examined portion of a subject, where the first IF signal is generated based on a first magnetic resonance signal received by the first local coil device from the subject; and a first receiving coupler including a first receiving coil array, the first receiving coil array including one or more first receiving coils and being located below a trajectory of the patient couch sliding over the magnetic resonance main body, and the first receiving coil array being configured to receive the first IF signal from the transmitting coil array in response to the transmitting coil array at least partially overlapping with the first receiving coil array.

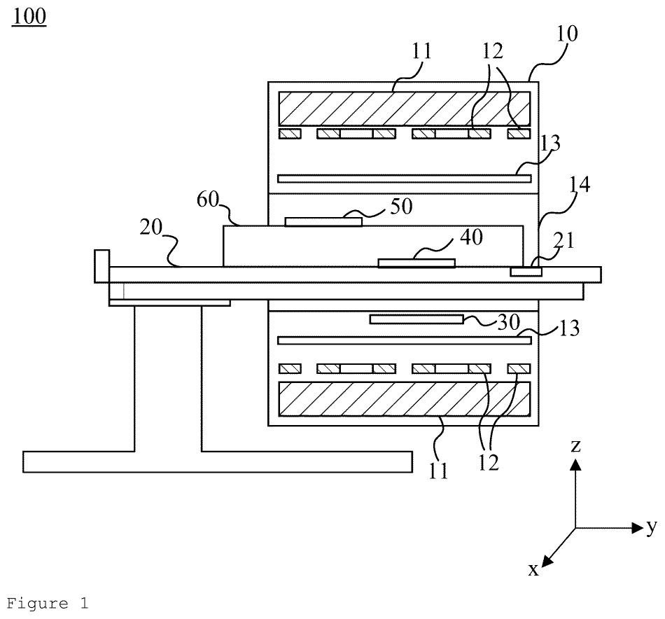

FIG. 1 shows a magnetic resonance imaging apparatus 100 according to an aspect of the present disclosure. According to some aspects, the magnetic resonance imaging apparatus 100 includes a magnetic resonance main body 10, a patient couch 20, a wireless transmission device, and a first local coil device 40. The wireless transmission device includes a main body-side transmission component 30 located on the side of the magnetic resonance main body 10 and a transmitting coupler 41 (not shown in FIG. 1) located on the side of the first local coil device 40.

According to some aspects, a magnetic resonance cavity 14 is provided in the magnetic resonance main body 10 to allow the patient couch 20 to slide into the same. The magnetic resonance cavity 14 extends through the magnetic resonance main body 10 in a longitudinal direction y. When the patient couch 20 carrying a subject 60 slides into the magnetic resonance cavity 14, magnetic resonance imaging may be performed on the subject 60.

According to some aspects, the magnetic resonance main body 10 has a field magnet 11 which generates a static magnetic field for aligning the nuclear spins of a portion of the subject 60 in an imaging area. According to some aspects, the imaging area of the magnetic resonance is the field of view (FOV) of the magnetic resonance, that is, the actual size of the image area in a frequency encoding direction and in a phase encoding direction.

According to some aspects, the magnetic resonance main body 10 further has a gradient coil 12 to superimpose variable magnetic fields in three spatial directions on the static magnetic field. According to some aspects, the gradient coil 12 is generally a coil made of an ordinary wire, which can generate fields orthogonal to each other.

According to some aspects, the magnetic resonance main body 10 further has a body coil 13, which is configured to: radiate a high-frequency signal input through a signal line into an examination volume, receive a magnetic resonance signal emitted by the subject 60, and output the magnetic resonance signal through the signal line. According to some aspects, the body coil 13 may be omitted, and a local coil (e.g., the first local coil device 40 in FIG. 1) may be used.

According to some aspects, the patient couch 20 may carry the subject 60 thereon and be driven to move in the longitudinal direction y and on a plane formed by the direction x and the direction y so as to move an examined portion of the subject 60 into the field of view of the magnetic resonance when examination is required, or to move the examined portion of the subject 60 out of the field of view of the magnetic resonance when the examination is finished.

According to some aspects, components of the wireless transmission device may be respectively arranged on the side of the magnetic resonance main body 10 and the side of the first local coil device 40 to realize wireless transmission between the side of the magnetic resonance main body 10 and the side of the first local coil device 40. According to some aspects, wireless transmission is signal transmission performed by wireless transmission technology, for example, wireless transmission performed by electromagnetic induction between coils as further described below. In the aspects described in the present disclosure, by realizing wireless transmission between the side of the magnetic resonance main body 10 and the side of the first local coil device 40, there is no need to use cables for signal transmission, thereby avoiding the various defects of signal transmission through cables mentioned above.

According to some aspects, the main body-side transmission component 30 of the wireless transmission device located on the side of the magnetic resonance main body 10 is arranged below a trajectory of the patient couch 20 sliding over the magnetic resonance main body 10. For example, the main body-side transmission component is arranged in an inner wall of the bottom of the magnetic resonance main body 10 and in the body coil 13 as shown in FIG. 1, or may be arranged above the inner wall of the bottom of the magnetic resonance main body 10, which is different from that shown in FIG. 1.

According to some aspects, parts of the main body-side transmission component 30 may be separately arranged at different positions. For example, as further described below, a first receiving coupler 31 of the wireless transmission device for wireless transmission with the first local coil device 40 is arranged in the magnetic resonance main body 10, and a second receiving coupler 32 of the wireless transmission device for wireless transmission with a second local coil device 50 is arranged on the inner wall of the magnetic resonance main body 10. For another example, the first receiving coupler 31 and the second receiving coupler 32 may be both arranged on the inner wall of the magnetic resonance main body 10, but they may be arranged at different positions on a plane where the patient couch 20 moves (that is, a plane formed by the x-axis and the y-axis in FIG. 1).

According to some aspects, the wireless transmission device includes a transmitting coupler 41 located in the first local coil device 40 and a first receiving coupler 31 located below the trajectory of the patient couch 20 sliding over the magnetic resonance main body 10. The transmitting coupler 41 performs wireless transmission with the first receiving coupler 31, for example, through electromagnetic induction. According to some aspects, the first local coil device 40 is located on the patient couch 20 and below the examined portion of the subject 60, such that a magnetic resonance signal from the examined portion of the subject 60 is received during imaging, and an IF signal is generated based on the magnetic resonance signal. According to some aspects, during the imaging, the transmitting coupler 41 located in the first local coil device 40 overlaps with the first receiving coupler 31, so that the IF signal is wirelessly transmitted. In this case, since the transmitting coupler 41 is in the first local coil device 40 located on the patient couch 20 and below the examined portion of the subject 60, a wireless transmission distance between the transmitting coupler 41 and the first receiving coupler 31 is only a distance in the z-axis direction in FIG. 1 (i.e., a direction perpendicular to the plane where the patient couch moves), which is approximately equal to the thickness of the patient couch 20.

According to some aspects, the transmitting coupler includes a transmitting coil array. The transmitting coil array includes one or more first transmitting coils arranged in the first local coil device and is configured to transmit a first IF signal, the first local coil device being located on the patient couch and below the examined portion of the subject. The first IF signal is generated based on a first magnetic resonance signal received by the first local coil device from the subject. Moreover, the first receiving coupler includes a first receiving coil array including one or more first receiving coils and located below the trajectory of the patient couch sliding over the magnetic resonance main body. The first receiving coil array is configured to receive the first IF signal from the transmitting coil array in response to the transmitting coil array at least partially overlapping with the first receiving coil array. According to some aspects, the first IF signal transmitted between the transmitting coil array of the first local coil device 40 and the first receiving coil array of the first receiving coupler of the wireless transmission device may be one first IF signal at one IF, or may be a plurality of first IF signals at a plurality of IFs. For example, the first IF signal may include two IF signals at 8.6 MHz and 11.4 MHz, respectively. According to some aspects, the number of transmitting coils in the transmitting coil array and the number of first receiving coils in the first receiving coil array may be associated with the number of IFs corresponding to the first IF signal. The more the number of IFs corresponding to the first IF signal, the fewer the number of transmitting coils in the transmitting coil array and the fewer the number of first receiving coils in the first receiving coil array.

According to some aspects, the number of first receiving coils in the first receiving coil array is set to be greater than the number of transmitting coils in the transmitting coil array, so that the area occupied by the first receiving coil array is larger, and the first IF signal can be transmitted between the transmitting coil array and the first receiving coil array as long as the transmitting coil array is in a certain range (rather than at a specific position).

According to some aspects, the term “overlapping” described in the present disclosure refers to overlapping in the plane where the patient couch 20 moves (i.e., the plane formed by the x-axis and the y-axis in FIG. 1). According to some aspects, the expression “at least partial overlapping” described in the present disclosure refers to partial overlapping or complete overlapping in the plane where the patient couch 20 moves (i.e., the plane formed by the x-axis and the y-axis in FIG. 1).

According to some aspects, the first receiving coil array overlaps with the FOV of the magnetic resonance main body so that the local coil (e.g., the first local imaging device 40) can overlap with the first receiving coil array during imaging, thereby performing wireless transmission between the two.

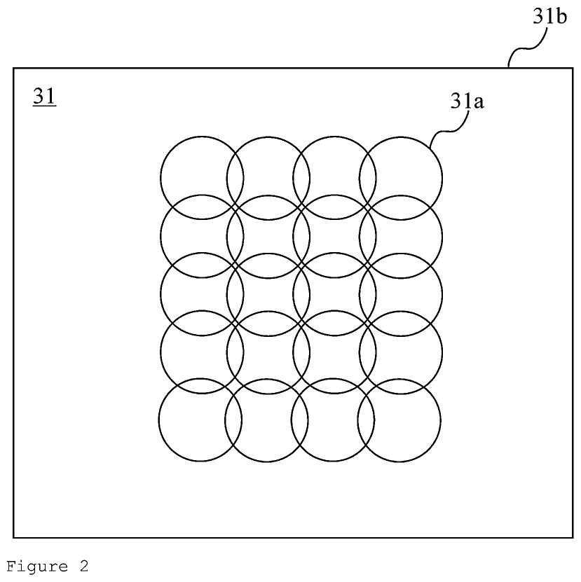

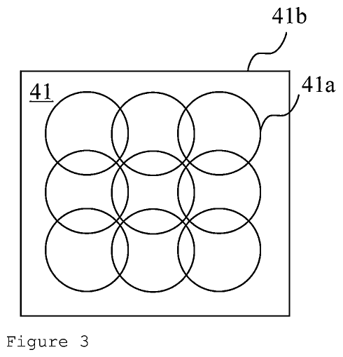

The first receiving coupler 31 and the transmitting coupler 41 of the wireless transmission device will be described in more detail below with reference to FIGS. 2 and 3.

FIG. 2 shows a structural diagram of the first receiving coupler 31 of the wireless transmission device according to the aspect of the present disclosure.

According to some aspects, the first receiving coupler 31 includes a first receiving coil array 31a. The first receiving coil array 31a includes one or more first receiving coils. According to some aspects, when the first receiving coil array 31a includes a plurality of first receiving coils, the plurality of first receiving coils in the first receiving coil array 31a are arranged in a two-dimensional plane and are arranged in a plurality of rows and/or a plurality of columns, for example, as shown in FIG. 2, arranged in 5 rows in the x-axis direction and in 4 columns in the y-axis direction, including (5×4)=20 coils. According to some aspects, adjacent coils in the first receiving coil array are arranged to partially overlap with each other to avoid strong coupling between the adjacent coils. According to some aspects, the first receiving coils in the first receiving coil array may be connected to the system via a preamplifier in a decoupled manner to avoid additional coupling generated due to induced current in these first receiving coils.

According to some aspects, the signal emitted by the subject 60 and detected by the first local coil device 40 may be converted into an IF signal, and the IF signal may be transmitted as a first IF signal to the first receiving coil array 31a in the main body-side transmission component 30 of the wireless transmission device. For example, a frequency converter is provided for each antenna coil, and the frequency converter may be configured to convert an original signal at a Larmor frequency into an IF signal that does not interfere with the original signal. According to some aspects, the frequency of the IF signal is lower than the frequency of the detected original signal emitted by the subject 60. According to some aspects, there may be a plurality of IF signals at different frequencies, for example, a first IF signal at 8.6 MHz and a second IF signal at 11.4 MHz.

According to some aspects, more than one original signal may be modulated onto one IF signal. For example, two original signals may be modulated onto one IF signal, and in the first receiving coil array shown in FIG. 2, 4×5=20 IF channel signals, which correspond to 40 original signals, may be received.

According to some aspects, in order to ensure that the signal-to-noise ratio (SNR) of wireless transmission between the first receiving coupler 31 on the side of the magnetic resonance main body 10 and the transmitting coupler 41 on the side of the first local coil device 40 is low, it should be ensured that a distance between the first receiving coil array 31a and the transmitting coil array 41a of the transmitting coupler 41 is less than half of the diameter of the first receiving coil array 31a during wireless transmission. For example, when the transmitting coil array 41a of the transmitting coupler 41 slides with the patient couch 20 to at least partially overlap with the first receiving coil array 31a, the distance between the transmitting coil array 41a and the first receiving coil array 31a is substantially the thickness of the patient couch 20. For example, for mechanical stability, the thickness of the patient couch 20 should be 5 cm, and therefore the diameters of the coils in the transmitting coil array 41a and the first receiving coil array 31a are not less than 10 cm. Therefore, the low SNR of wireless transmission can be ensured by setting the diameters of the coils according to the distance between the transmitting coil array 41a and the first receiving coil array 31a. Furthermore, since the transmitting coil array 41a and the first receiving coil array 31a are as close as possible to each other during wireless transmission, that is, they are separated by only the thickness of the patient couch, the coils in the transmitting coil array 41a and the first receiving coil array 31a do not need to be set too large, thereby reducing the sizes of the transmitting coil array 41a and the first receiving coil array 31a.

According to some aspects, in the first local coil device 40, in order to mix the signal emitted by the subject 60, a local oscillator (LO) signal is required. According to some aspects, in order to provide a stable LO signal, the LO signal is generated on the side of the magnetic resonance main body 10, and the LO signal is transmitted into the first local coil device 40 through the wireless transmission device. According to some aspects, a power signal is wirelessly transmitted to the first local coil device 40 through the wireless transmission device, thereby simplifying a power supply component (e.g., a battery for power supply) on the side of the first local coil device 40. According to some aspects, a tuning control signal is wirelessly transmitted to the first local coil device 40 through the wireless transmission device, thereby realizing the tuning or detuning of the first local coil device 40. According to some aspects, the first local coil device 40 can be selectively turned on or off by means of the tuning control signal. Specifically, the magnetic resonance coil in the first local coil device 40 is tuned to a specific high-frequency signal after receiving a tuning signal so as to be activated to receive a signal returned from the subject, and the magnetic resonance coil in the first local coil device 40 will no longer respond to the signal returned from the subject after receiving a detuning signal.

According to some aspects, a corresponding first LO signal may be configured for each first IF signal. For example, two first LO signals, respectively, at 55 MHz and 75 MHz, are configured for two first IF signals, respectively, at 8.6 MHz and 11.4 MHz. According to some other aspects, a common first LO signal may be configured for a plurality of first IF signals. For example, only one first LO signal is transmitted to the first local coil device 40 through the wireless transmission device, and the local coil device 40 may generate signals at more frequencies based on the first LO signal (e.g., via a phase-locked loop or other components) to perform frequency mixing corresponding to the plurality of first IF signals.

According to some aspects, the first receiving coupler 31 further includes a first receiving auxiliary coil 31b. The first receiving coil array 31a is arranged in the first receiving auxiliary coil 31b. According to some aspects, by the first receiving coil array 31a being arranged in the first receiving auxiliary coil 31b, it is meant that the first receiving coil array 31a is within the range of the first receiving auxiliary coil 31b on the plane where the patient couch moves (i.e., the plane formed by the x-axis and the y-axis). According to some aspects, the first receiving coil array 31a and the first receiving auxiliary coil 31b are arranged to be coplanar. According to some aspects, the first receiving coil array 31a and the first receiving auxiliary coil 31b may alternatively be arranged to be concentric. According to some aspects, the first receiving auxiliary coil 31b should be large enough so that when the first local coil device 40 moves away from the FOV but is still close to the FOV, it can still receive the power signal and the tuning control signal and be controlled to be detuned.

According to some aspects, the first receiving auxiliary coil 31b is configured to transmit at least one of a first power signal, a first tuning control signal, and a first LO signal.

According to some aspects, the transmitting coupler 41 includes a transmitting auxiliary coil 41b in addition to the transmitting coil array 41a. The transmitting coil array 41a is arranged in the transmitting auxiliary coil 41b. According to some aspects, by the transmitting coil array 41a being arranged in the transmitting auxiliary coil 41b, it is meant that the transmitting coil array 41a is within the range of the transmitting auxiliary coil 41b on the plane where the patient couch moves (i.e., the plane formed by the x-axis and the y-axis). According to some aspects, the transmitting coupler 41 is configured to perform at least one of the following: the transmitting auxiliary coil 41b receiving at least one of the first power signal and the first tuning control signal from the first receiving auxiliary coil 31b in response to the transmitting auxiliary coil 41b at least partially overlapping with the first receiving auxiliary coil 31b; and a component of the transmitting coupler 41 that is configured to transmit the first LO signal receiving the first LO signal from the first receiving auxiliary coil 31b in response to the component of the transmitting coupler 41 that is configured to transmit the first LO signal at least partially overlapping with the first receiving auxiliary coil 31b. The component of the transmitting coupler 41 that is configured to transmit the first LO signal is the transmitting coil array 41a or the transmitting auxiliary coil 41b.

FIG. 3 shows a structural diagram of the transmitting coupler 41 of the wireless transmission device according to the aspect of the present disclosure.

According to some aspects, the transmitting coupler 41 includes a transmitting coil array 41a. The transmitting coil array 41a includes one or more first transmitting coils. According to some aspects, the arrangement of the first transmitting coils in the transmitting coil array 41a may be similar to the arrangement of the first receiving coils in the first receiving coil array 31a, but the difference is that the number of first transmitting coils in the transmitting coil array 41a is less than the number of first receiving coils in the first receiving coil array 31a (e.g., the number of rows and/or columns of the transmitting coil array 41a is fewer), so that the transmitting coil array 41a can perform wireless transmission without being precisely at a specific position, but can perform wireless transmission within a certain range.

According to some aspects, the transmitting coil array 41a is configured to transmit the first IF signal to the first receiving coil array 31a in response to the transmitting coil array 41a at least partially overlapping with the first receiving coil array 31a.

According to some aspects, the transmitting coil array 41a is further configured to receive the first LO signal from the first receiving auxiliary coil 31b in response to the transmitting coil array 41a at least partially overlapping with the first receiving auxiliary coil 31b.

According to some aspects, the transmitting coupler 41 further includes a transmitting auxiliary coil 41b. The transmitting coil array 41a is arranged in the transmitting auxiliary coil 41b, and the transmitting auxiliary coil 41b is configured to receive at least one of the first power signal, the first tuning control signal and the first LO signal from the first receiving auxiliary coil 31b in response to the transmitting auxiliary coil 41b at least partially overlapping with the first receiving auxiliary coil 31b. It should be understood that, as described above, either the transmitting coil array 41a or the transmitting auxiliary coil 41b in the transmitting coupler 41 can be used to receive the first LO signal from the first receiving auxiliary coil 31b.

According to some aspects, the transmitting auxiliary coil 41b is further configured to transmit a signal corresponding to an induced voltage to the main body-side transmission component 30, allowing the main body-side transmission component 30 to adjust the transmitted power signal such that a required voltage value is reached on the side of the first local coil device 40. According to some aspects, the transmitting coil array 41a and the transmitting auxiliary coil 41b are arranged to be coplanar. According to some aspects, the transmitting coil array 41a and the transmitting auxiliary coil 41b may alternatively be arranged to be concentric.

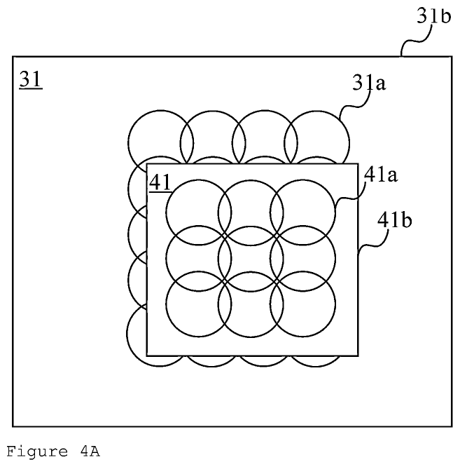

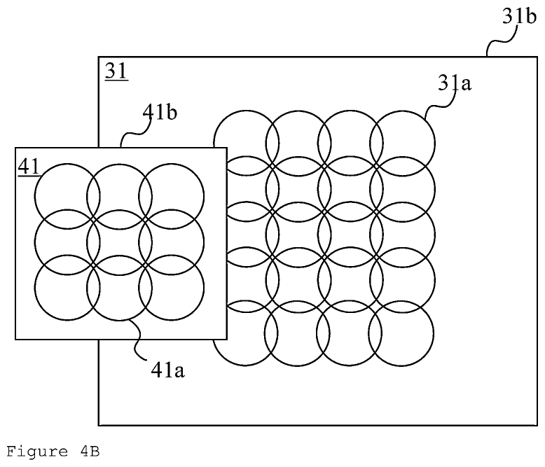

FIGS. 4A-4B show schematic diagrams of wireless transmission between the first receiving coupler 31 and the transmitting coupler 41 according to the aspect of the present disclosure. It should be understood that the first receiving coupler 31 of the main body-side transmission component 30 is generally below the patient couch 20, and the transmitting coupler 41 of the first local coil device 40 is generally above the patient couch 20 so that the two are separated by at least the patient couch 20, but for the convenience of illustration, only the first receiving coupler 31 and the transmitting coupler 41 are drawn, and other components of the magnetic resonance imaging apparatus 100 are omitted.

As shown in FIG. 4A, when the transmitting coupler 41 moves to a first position, the corresponding first local coil device 40 (in which other components except the transmitting coupler 41 are not shown) also moves into the Field Of View (FOV), so that the first magnetic resonance signal emitted by the subject 60 can be received, and the transmitting coil array 41a at least partially overlaps with the first receiving coil 31a, so that the corresponding first IF signal can be wirelessly transmitted between the transmitting coil array 41a and the first receiving coil 31a. In addition, as also shown in FIG. 4A, the transmitting auxiliary coil 41b located in the first local coil device 40 at least partially overlaps with the first receiving auxiliary coil 31b, so that the first power signal and/or the first tuning control signal are transmitted between the transmitting auxiliary coil 41b and the first receiving auxiliary coil 31b, and the transmitting coil array 41a or the transmitting auxiliary coil 41b at least partially overlaps with the first receiving auxiliary coil 31b, so that the first LO signal is transmitted between the transmitting coil array 41a or the transmitting auxiliary coil 41b and the first receiving auxiliary coil 31b. It should be noted that the transmitting coupler 41 only needs to be above the first receiving coupler 31 and does not have to be moved to be concentric with the first receiving coupler 31.

As shown in FIG. 4B, when the transmitting coupler 41 moves to a second position, the corresponding first local coil device 40 (in which other components except the transmitting coupler 41 are not shown) also moves out of the FOV, so that the subject 60 is no longer imaged, and the transmitting coil array 41a no longer overlaps with the first receiving coil 31a, so that the transmitting coil array 41a no longer transmits the first IF signal to the first receiving coil 31a. In addition, as also shown in FIG. 4B, there is still an overlapping portion between the transmitting auxiliary coil 41b and the first receiving auxiliary coil 31b, and the transmitting auxiliary coil 41b still receives a power signal from the first receiving auxiliary coil 31b and a tuning control signal corresponding to detuning, so that the first local coil device 40 is detuned.

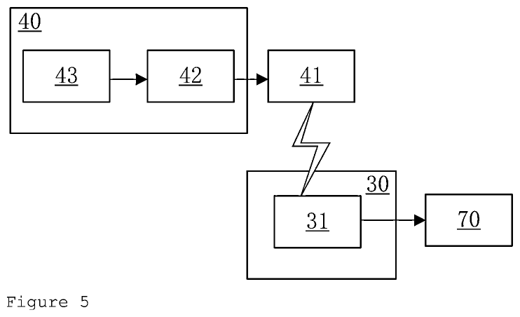

FIG. 5 shows a schematic diagram of transmission of the first IF signal in the wireless transmission device according to the aspect of the present disclosure.

As shown in FIG. 5, the first local coil device 40 includes a first magnetic resonance coil 43 and a first frequency converter 42, and the wireless transmission device includes a transmitting coupler 41 and a first receiving coupler 31.

As described above, the first magnetic resonance coil 43 is configured to detect a first magnetic resonance signal emitted by the subject 60, and the first frequency converter 42 converts the first magnetic resonance signal into a first IF signal. According to some aspects, a low noise amplifier may be provided between the first magnetic resonance coil 43 and the first frequency converter 42 to amplify the first magnetic resonance signal, and a matching circuit may be provided between the first magnetic resonance coil 43 and the low noise amplifier such that the impedance of the first magnetic resonance coil 43 is adapted to the low noise amplifier. According to some aspects, the transmitting coupler 41 is coupled with the first receiving coupler 31 to wirelessly transmit the first IF signal converted by the first frequency converter 42 to the first receiving coupler 31. According to some aspects, the first receiving coupler 31 transmits the received first IF signal to a control terminal 70 of the magnetic resonance imaging apparatus. The main body-side transmission component 30 may further include other components (e.g., an amplifier, and an ADC converter) to post-process the first IF signal, and transmit the post-processed signal to the control terminal 70.

According to some aspects, the first local coil device 40 is a local coil device having a contact surface with the patient couch 20, and the first local coil device 40 is a head coil device, a neck coil device, a head and neck coil device, a spine coil device, a chest coil device, a knee coil device, or an ankle coil device.

In practical applications, there are also some local coil devices (e.g., body coil devices or flexible coil devices) that have no contact surface with the patient couch, and the distance between them and the main body-side transmission component 30 is relatively far. Therefore, if wireless transmission in the wireless transmission device described with reference to the first local coil device 40 is adopted, the SNR may be too high due to the long coupling distance. Therefore, a local coil device having no contact surface with the patient couch 20 may be connected to a local coupler (e.g., a local coupler arranged on the patient couch 20) in a wired manner, and then perform wireless transmission with the main body-side transmission component 30 through the local coupler, thereby shortening the coupling distance of the wireless transmission.

According to some aspects, the magnetic resonance imaging apparatus further includes a second local coil device. The second local coil device includes: a second magnetic resonance coil configured to detect a second magnetic resonance signal emitted by the subject; and a second frequency converter configured to: convert the second magnetic resonance signal into a second intermediate frequency signal; and transmit the second intermediate frequency signal to a local coupler in response to the second local coil device being electrically connected to the local coupler.

According to some aspects, the wireless transmission device further includes: a local coupler including a local coupling coil array, the local coupling coil array including one or more local coupling coils, and being configured to be electrically connected to a second local coil device located above the examined portion of the subject to receive a second IF signal from the second local coil device, and to transmit the second IF signal, where the second IF signal is generated based on a second magnetic resonance signal received by the second local coil device from the subject; and a second receiving coupler including a second receiving coil array, the second receiving coil array including one or more second receiving coils and being located below the trajectory of the patient couch sliding over the magnetic resonance main body, and the second receiving coil array being configured to receive the second IF signal from the local coupling coil array in response to the local coupling coil array at least partially overlapping with the second receiving coil array.

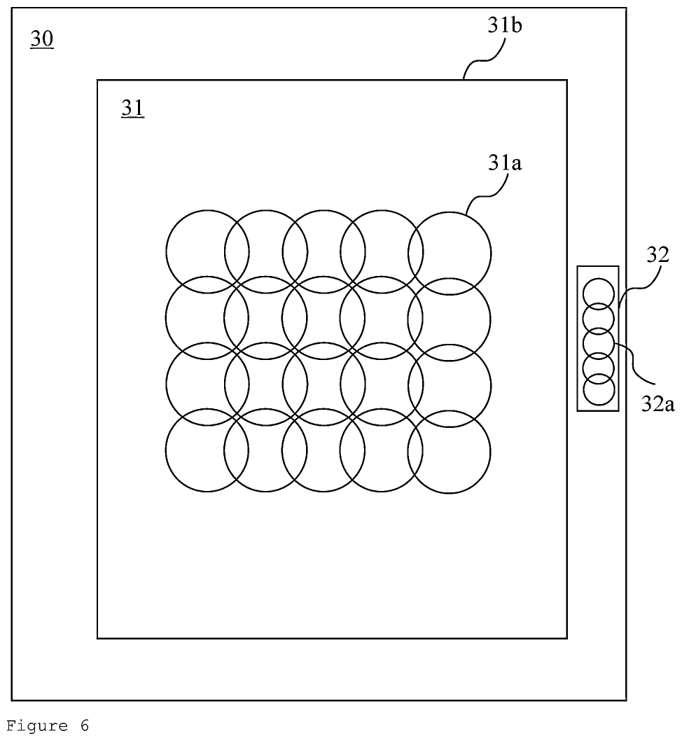

FIG. 6 shows a structural diagram of the main body-side transmission component 30 of the wireless transmission device according to the aspect of the present disclosure. As shown in FIG. 6, the main body-side transmission component 30 further includes a second receiving coupler 32 in addition to a first receiving coupler 31. The second receiving coupler 32 includes a second receiving coil array 32a, including one or more second receiving coils. The second coil array 32a is configured to receive a second IF signal from the local coupling coil array 21a in response to the local coupling coil array 21a of the local coupler 21 electrically connected to the second local coil device 50 at least partially overlapping with the second receiving coil array 32a. The second IF signal is generated based on a second magnetic resonance signal emitted by the subject 60 and detected by the second local coil device, and the local coupling coil array includes one or more local coupling coils. According to some aspects, the first receiving coupler 31 in FIG. 6 may be the same as or similar to the first receiving coupler 31 described with reference to FIG. 2. According to some aspects, as described below, when the second receiving coupler 32 is locked to the patient couch 20, the second receiving coil array 32a at least partially overlaps with the local coupling coil array 21a, and the second receiving coil array 32a receives the second IF signal from the local coupling coil array 21a.

According to some aspects, the second IF signal transmitted between the local coupling coil array 21a of the local coupler 21 and the second receiving coil array 32a of the second receiving coupler 32 may be one second IF signal at one IF, or a plurality of second IF signals at a plurality of IFs. For example, the second IF signal may include two IF signals at 8.6 MHz and 11.4 MHz, respectively. According to some aspects, the number of local coupling coil arrays 21a in the local coupler 21 and the number of second receiving coil arrays 32a in the second receiving coupler 32 may be associated with the number of IFs corresponding to the second IF signal. The more the number of IFs corresponding to the second IF signal, the fewer the number of local coupling coil arrays 21a in the local coupler 21 and the fewer the number of second receiving coil arrays 32a in the second receiving coupler 32.

According to some aspects, since the local coupler 21 and the second receiving coupler 32 may be locked for wireless transmission, the number and/or arrangement of the local coupling coil arrays 21a in the local coupler 21 may be set to be the same as those of the second receiving coil arrays 32a in the second receiving coupler 32.

According to some aspects, the first receiving coupler 31 and the second receiving coupler 32 may be arranged at different positions relative to the magnetic resonance main body 10. For example, the first receiving coupler 31 may be arranged in the inner wall of the magnetic resonance main body 10 to reduce the number of components exposed to the outside, and the second receiving coupler 32 may be arranged on the inner wall of the magnetic resonance main body 10 or connected to the magnetic resonance main body 10 via a cable, etc., so that the second receiving coupler 32 can be locked to the local coupler when the patient couch 20 slides into the cavity 14, to reduce a coupling distance between the second receiving coupler 32 and the local coupler. According to some other aspects, the first receiving coupler 31 and the second receiving coupler 32 may both be arranged on the inner wall of the magnetic resonance main body 10.

According to some aspects, the local coupler may be arranged on the patient couch 20, for example, in a slot on a left side of, on a right side of or near a head position of the patient couch 20. According to some other aspects, the local coupler may be a component provided separately from the patient couch 20. For example, the local coupler may be connected to the second local coil device 50 on the subject 60 via a cable passing through an opening on the patient couch 20, and the local coupler is located below the patient couch 20 and may be locked to the second receiving coupler 32.

According to some aspects, the second receiving coupler 32 may be arranged outside (e.g., on the left or right side of) the FOV or in the FOV of the magnetic resonance main body, and accordingly, the local coupler on the patient couch 20 is arranged in a slot on the left side of, on the right side of or near the head position of the patient couch 20. According to some aspects, since the local coupler on the patient couch 20 cannot be configured to be too large, the coupling distance between the second receiving coupler 32 and the local coupler should be set to be relatively small. For example, due to the limited space on the patient couch, the diameter of a coil of the local coupler may be set to be 15 mm, and the corresponding coupling distance should be less than 7.5 mm. According to some aspects, one or more second receiving couplers 32 may be provided in the main body-side transmission component 30. For example, one second receiving coupler 32 may be provided on either side of the first receiving coupler 31.

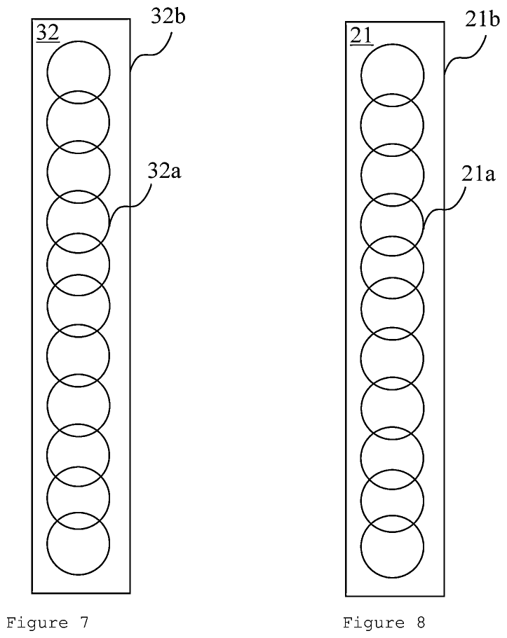

Hereinafter, structural diagrams of the second receiving coupler 32 of the main body-side transmission component 30 and the local coupler 21 according to the aspect of the present disclosure are described in detail with reference to FIGS. 7 and 8.

According to some aspects, as shown in FIG. 7, the second receiving coil array 32a in the second receiving coupler 32 is configured as a column of second receiving coils extending in a direction along two sides of the patient couch (e.g., the direction y shown in FIG. 1). It should be understood that the second receiving coil array 32a in FIG. 7 is only schematic, and the second receiving coil array 32a may have a different number of second receiving coils. According to some aspects, adjacent second receiving coils in the second receiving coil array 32 may be configured to partially overlap with each other.

According to some aspects, similar to the description with reference to the first receiving coupler 31, the second receiving coupler 32 receives a second LO signal from the local coupler 21 to implement conversion from the second magnetic resonance signal into the second LO signal. According to some aspects, similar to the description with reference to the first receiving coupler 31, the second receiving coupler 32 receives one second LO signal at one frequency or a plurality of second LO signals at a plurality of frequencies from the local coupler 21.

According to some aspects, the second receiving coupler 32 further includes a second receiving auxiliary coil 32b. The second receiving coil array 32a is arranged in the second receiving auxiliary coil 32b, and the second receiving auxiliary coil 32b is configured to transmit at least one of a second power signal, a second tuning control signal and a second LO signal. According to some aspects, by the second receiving coil array 32a being arranged in the second receiving auxiliary coil 32b, it is meant that the second receiving coil array 32a is within the range of the second receiving auxiliary coil 32b on the plane where the patient couch moves (i.e., the plane formed by the x-axis and the y-axis). According to some aspects, the second receiving coil array 32a and the second receiving auxiliary coil 32b are arranged to be coplanar, and/or the second receiving coil array 32a and the second receiving auxiliary coil 32b are arranged to be concentric.

According to some aspects, the second receiving auxiliary coil 32b is configured to perform at least one of the following: transmitting the second power signal and/or the second tuning control signal to a coupling auxiliary coil 21b of the local coupler 21 in response to the coupling auxiliary coil 21b of the local coupler 21 at least partially overlapping with the second receiving auxiliary coil 32b; and transmitting a second LO signal to a component of the local coupler 21 that is configured to transmit the second LO signal in response to the component configured to transmit the second LO signal at least partially overlapping with the second receiving auxiliary coil 32b. The component of the local coupler that is configured to transmit the second LO signal is the local coupling coil array 21a or the coupling auxiliary coil 21b. According to some aspects, as described below, when the second receiving coupler 32 is locked to the patient couch 20, the coupling auxiliary coil 21b at least partially overlaps with the second receiving auxiliary coil 32b, the coupling auxiliary coil 21b receives the second power signal and/or the second tuning control signal from the second receiving auxiliary coil 32b, the local coupling coil array 21a or the coupling auxiliary coil 21b at least partially overlaps with the second receiving auxiliary coil 32b, and the local coupling coil array 21a or the coupling auxiliary coil 21b receives the second LO signal from the second receiving auxiliary coil 32b.

According to some aspects, the second local coil device 50 of the magnetic resonance imaging apparatus 100 is configured to: detect a second magnetic resonance signal emitted by the subject 60; convert the second magnetic resonance signal into a second IF signal; and transmit the second IF signal to the second local coil device 50 in response to the second local coil device 50 being electrically connected to the local coupler 21.

According to some aspects, the local coupler 21 of the magnetic resonance imaging apparatus 100 includes a local coupling coil array 21a. The local coupling coil array 21a includes one or more local coupling coils, and the local coupling coil array 21a is configured to: transmit the second IF signal to the second receiving coil array 32a in response to the local coupling coil array 21a being electrically connected to the second local coil device 50 and the local coupling coil array 21a at least partially overlapping with the second receiving coil array 32a of the main body-side transmission component 30.

According to some aspects, similar to what is described above with reference to the first local coil device 40, a second magnetic resonance coil and a second frequency converter may be provided in the second local coil device 50. The second magnetic resonance coil is configured to detect a second magnetic resonance signal emitted by the subject 60, and the second frequency converter is configured to convert the second magnetic resonance signal into the second IF signal. According to some aspects, similar to what is described above with reference to the first local coil device 40, a low noise amplifier and/or a matching circuit may be provided between the second magnetic resonance coil and the second frequency converter.

FIG. 8 shows a structural diagram of a local coupler 21 according to the aspect of the present disclosure. According to some aspects, the structure of the local coupler 21 in FIG. 8 is the same as the structure of the second receiving coupler 32 in FIG. 7. According to some aspects, when wireless transmission is performed between the local coupler 21 and the second receiving coupler 32, the local coupler 21 and the second receiving coupler 32 may be locked to each other to reduce the coupling distance.

According to some aspects, the local coupling coil array 21a is further configured to transmit the second IF signal to the second receiving coil array 32a in response to the local coupling coil array 21a at least partially overlapping with the second receiving coil array 32a. According to some aspects, the local coupler 21 further includes a coupling auxiliary coil 21b. The local coupling coil array 21a is arranged in the coupling auxiliary coil 21b. According to some aspects, by the local coupling coil array 21a being arranged in the coupling auxiliary coil 21b, it is meant that the local coupling coil array 21a is within the range of the coupling auxiliary coil 21b on the plane where the patient couch moves (i.e., the plane formed by the x-axis and the y-axis). According to some aspects, the local coupler 21 is configured to perform at least one of the following: the coupling auxiliary coil 21b receiving at least one of the second power signal and the second tuning control signal from the second receiving auxiliary coil 32b in response to the coupling auxiliary coil 21b at least partially overlapping with the second receiving auxiliary coil 32b; and a component of the local coupler 21 that is configured to transmit the second LO signal receiving the second LO signal from the second receiving auxiliary coil 32b in response to the component of the local coupler 21 that is configured to transmit the second LO signal at least partially overlapping with the second receiving auxiliary coil 32b. The component of the local coupler 21 that is configured to transmit the second LO signal is the local coupling coil array 21a or the coupling auxiliary coil 21b.

According to some aspects, as described with reference to the transmitting auxiliary coil 41b, the coupling auxiliary coil 21b is further configured to transmit a signal corresponding to an induced voltage to the main body-side transmission component 30, allowing the main body-side transmission component 30 to adjust the transmitted power signal such that a required voltage value is reached on the side of the second local coil device 40.

According to some aspects, the wireless transmission device further includes a locking structure. The locking structure is configured such that when the patient couch 20 slides to a locking position, the locking structure locks the second receiving coupler 32 to the local coupler 21; and when the patient couch slides to an unlocking position, the locking structure unlocks the second receiving coupler 32 from the local coupler 21. According to some aspects, the locking position is a position where the patient couch 20 slides into the magnetic resonance cavity 14 and is about to image the subject 60 by means of the second local coil device 50. According to some aspects, the unlocking position is a position where the patient couch 20 finishes imaging the subject 60 by means of the second local coil device 50 and slides out of the magnetic resonance cavity 14. According to some aspects, the locking mechanism locks the second receiving coupler 32 to overlap with the local coupler 21 for performing wireless transmission therebetween.

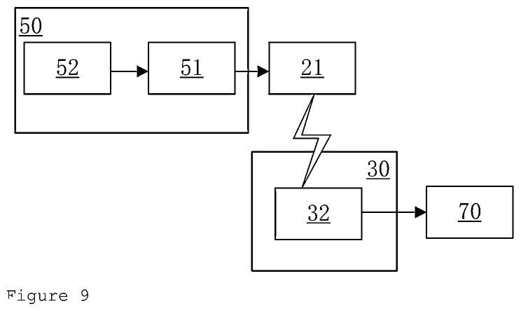

FIG. 9 shows a schematic diagram of transmission of the second IF signal in the wireless transmission device according to the aspect of the present disclosure.

As described above, the second local coil device 50 is electrically connected to the local coupler 21, and the local coupler 21 performs wireless transmission with the second receiving coupler 32. As shown in FIG. 9, the second local coil device 50 includes a second magnetic resonance coil 52 and a second frequency converter 51, and the main body-side transmission component 30 includes the second receiving coupler 32.

According to some aspects, the second magnetic resonance coil 52 is configured to detect a second magnetic resonance signal emitted by the subject 60, and the second frequency converter 51 converts the second magnetic resonance signal into the second IF signal. According to some aspects, a low noise amplifier may be provided between the second magnetic resonance coil 52 and the second frequency converter 51 to amplify the second magnetic resonance signal, and a matching circuit may be provided between the second magnetic resonance coil 52 and the low noise amplifier such that the impedance of the second magnetic resonance coil 52 is adapted to the low noise amplifier. According to some aspects, the second local coil device 50 (e.g., the second frequency converter 51 thereof) is electrically connected to the local coupler 21 to transmit the second IF signal converted by the second frequency converter 51 to the local coupler 21. According to some aspects, the local coupler 21 is coupled with the second receiving coupler 32 of the main body-side transmission component 30 to wirelessly transmit the second IF signal to the second receiving coupler 32. According to some aspects, the second receiving coupler 32 transmits the received second IF signal to a control terminal 70 of the magnetic resonance imaging apparatus. The main body-side transmission component 30 may further include other components (e.g., an amplifier, and an ADC converter) to post-process the second IF signal, and transmit the post-processed signal to the control terminal 70.

According to some aspects, the second local coil device 50 is a local coil device having no contact surface with the patient couch 20, and the second local coil device 50 is a body coil device or a flexible coil device.

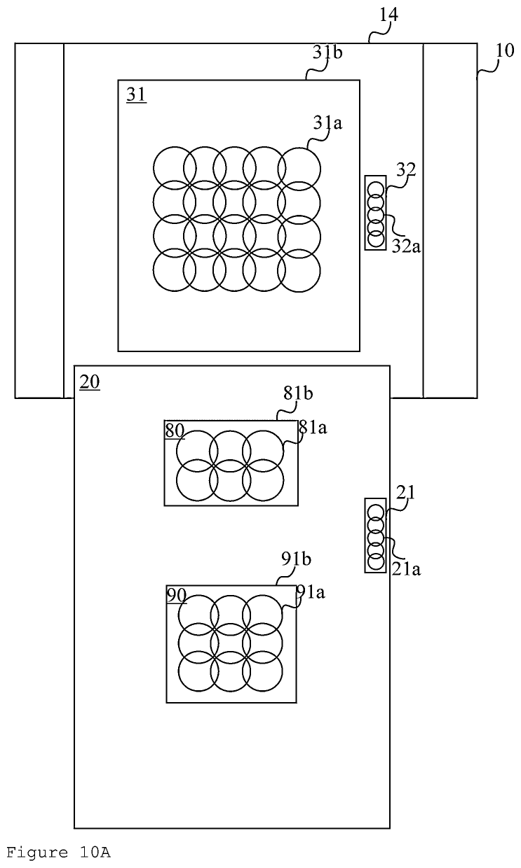

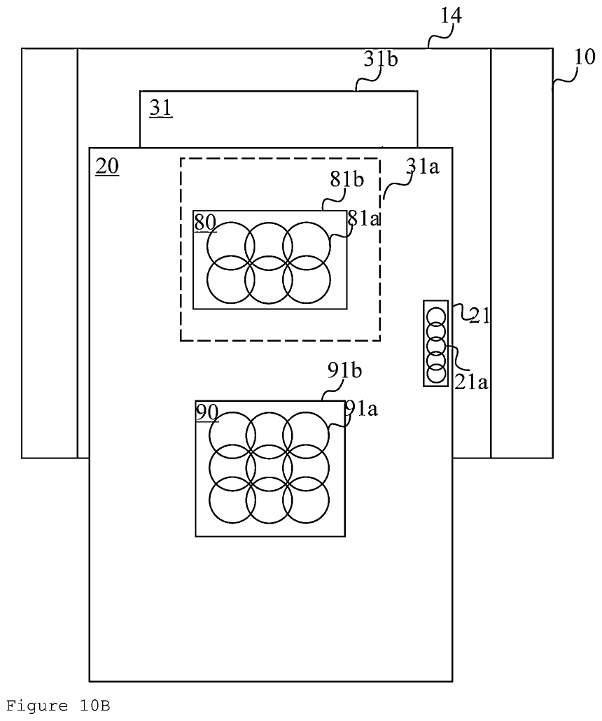

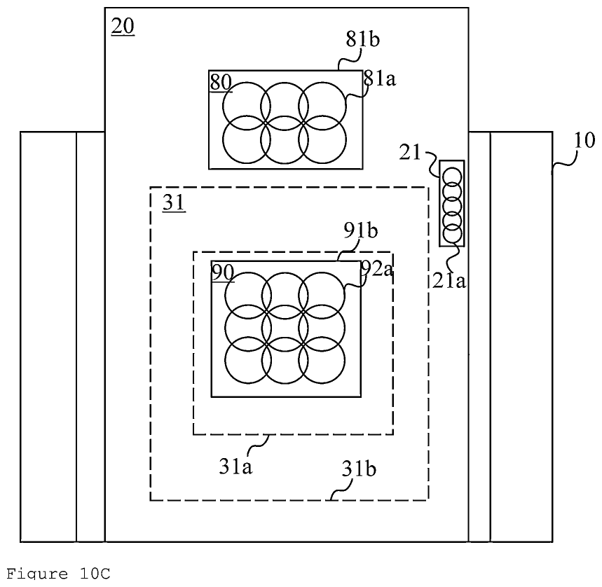

FIGS. 10A-10C show schematic diagrams of application scenarios of operating a magnetic resonance imaging apparatus 100 according to an aspect of the present disclosure. Two first local coil devices, namely, a head coil device 80 and a spine coil device 90, are placed on the patient couch 20, and the magnetic resonance imaging apparatus 100 may further include a local coupler 21 arranged in the patient couch 20. In FIGS. 10B and 10C, for the sake of clarity, only the outline of a first receiving coil array 31a is schematically drawn in a rectangle, and the details of coils thereof are not drawn. However, it should be understood that the structure of the first receiving coil array 31a in FIGS. 10B and 10C is the same as that of the first receiving coil array 31a in FIG. 10A.

As shown in FIG. 10A, the patient couch 20 begins to move into the cavity 14 in the magnetic resonance main body 10. None of the local coil devices (e.g., a head coil device 80, a spine coil device 90, and a second local coil device that can be connected to the local coupler 21) is moved into the FOV, neither of the head coil device 80 and the spine coil device 90 overlaps with the first receiving coupler 31 of the main body-side transmission component 30, and the second receiving coupler 32 is not locked to the patient couch 20. Accordingly, the head coil device 80, the spine coil device 90, and the second local coil device that can be connected to the local coupler 21 are not used to image the subject (not drawn in the figures for the sake of simplicity), and also do not perform wireless transmission with the main body-side transmission component 30.

As shown in FIG. 10B, the patient couch 20 moves into the cavity 14 in the magnetic resonance main body 10. The head coil device 80 moves into the FOV, while the spine coil device 90 has not yet moved into the FOV. Accordingly, the head of the subject can be imaged by means of the head coil device 80, while the spine of the subject has not yet been imaged by means of the spine coil device 90. The head coil device 80 detects a magnetic resonance signal emitted by the head of the subject. In addition, since a head transmitting coil array 81a of the head coil device 80 overlaps with the first receiving coil array 31a, and the head transmitting coil array 81a and a head transmitting auxiliary coil 81b of the head coil device 80 overlap with a first receiving auxiliary coil 31b, the head coil device 80 transmits an IF signal, into which the magnetic resonance signal emitted by the head of the subject is converted, to the main body-side transmission component 30. In addition, the second receiving coupler 32 has been locked to the patient couch 20, and a second local coil device having no contact surface with the patient couch 20 may also be electrically connected to the local coupler 21 on the patient couch 20, so that imaging is performed by means of the second local coil device and a second IF signal is transmitted to the second receiving coupler 32.

As shown in FIG. 10C, the patient couch 20 moves further. The head coil device 80 moves out of the FOV, and the spine coil device 90 moves into the FOV. Accordingly, the imaging of the head of the subject by means of the head coil device 80 can be finished, and the spine of the subject can be imaged by means of the spine coil device 90. The spine coil device 80 detects a magnetic resonance signal emitted by the spine of the subject. In addition, since a spine transmitting coil array 91a of the spine coil device 90 overlaps with the first receiving coil array 31a, and the spine transmitting coil array 91a and a spine transmitting auxiliary coil 91b of the spine coil device 90 overlap with the first receiving auxiliary coil 91b, the spine coil device 90 transmits an IF signal, into which the magnetic resonance signal emitted by the spine of the subject is converted, to the main body-side transmission component 30. In addition, the second receiving coupler 32 is unlocked from the patient couch 20, and wireless transmission is no longer performed between the local coupler 21 and the second receiving coupler 32.

As can be seen from FIGS. 10A-10C, by moving the patient couch 20, the local coil device (e.g., the head coil device 80, or the spine coil device 90) having a contact surface with the patient couch 20 can be moved into the FOV in sequence to image the corresponding portion, and overlap with the first receiving coupler 31 to perform wireless signal transmission; and, by electrically connecting the local coil device having no contact surface with the patient couch 20 to the local coupler 21, the patient couch 20 can be moved to the locking position so that the local coupler 21 is locked to the second receiving coupler 32 to perform wireless signal transmission.

It should be understood that the above application scenarios are only illustrative, and the actual application scenarios may be different from this. For example, the unlocking position of the patient couch 20 may be set differently from that in FIG. 10C, and the patient couch 20 may be unlocked only when it slides to a position farther from the magnetic resonance cavity 10 than the position in FIG. 10C.

Although aspects or examples of the present disclosure have already been described with reference to the drawings, it should be understood that the methods, systems and devices described above are merely exemplary aspects or examples, and the scope of the present invention is not limited by these aspects or examples, instead being defined solely by the granted claims and the equivalent scope thereof. Various key elements in the aspects or examples may be omitted or may be replaced by equivalent key elements thereof. In addition, the steps may be executed in an order different from that described in the present disclosure. Furthermore, various key elements in the aspects or examples may be combined in various ways. Importantly, as technology evolves, many key elements described here may be replaced by equivalent key elements appearing after the present disclosure.

1. A wireless transmission device for a magnetic resonance imaging apparatus, comprising:
a transmitting coupler comprising a transmitting coil array, the transmitting coil array comprising one or more first transmitting coils arranged in a first local coil device and being configured to transmit a first intermediate frequency signal, the first local coil device being located on a patient couch and below an examined portion of a subject, wherein the first intermediate frequency signal is generated based on a first magnetic resonance signal received by the first local coil device from the subject; and
a first receiving coupler comprising a first receiving coil array, the first receiving coil array comprising one or more first receiving coils and being located below a trajectory of the patient couch sliding over a magnetic resonance main body, and the first receiving coil array being configured to receive the first intermediate frequency signal from the transmitting coil array in response to the transmitting coil array at least partially overlapping with the first receiving coil array.

2. The wireless transmission device of claim 1, wherein the first receiving coil array overlaps with a field of view of the magnetic resonance main body.

3. The wireless transmission device of claim 1,
wherein the first receiving coupler further comprises a first receiving auxiliary coil, wherein the first receiving coil array is arranged in the first receiving auxiliary coil, and the first receiving auxiliary coil is configured to transmit at least one of a first power signal, a first tuning control signal, and a first local oscillator signal, and
the transmitting coupler further comprises a transmitting auxiliary coil, wherein the transmitting coil array is arranged in the transmitting auxiliary coil, and the transmitting coupler is configured to perform:
the transmitting auxiliary coil receiving at least one of the first power signal and the first tuning control signal from the first receiving auxiliary coil in response to the transmitting auxiliary coil at least partially overlapping with the first receiving auxiliary coil; or
a component of the transmitting coupler that is configured to transmit the first local oscillator signal receiving the first local oscillator signal from the first receiving auxiliary coil in response to the component of the transmitting coupler that is configured to transmit the first local oscillator signal at least partially overlapping with the first receiving auxiliary coil, wherein the component of the transmitting coupler that is configured to transmit the first local oscillator signal is the transmitting coil array or the transmitting auxiliary coil.

4. The wireless transmission device according to claim 1, further comprising:
a local coupler comprising a local coupling coil array, the local coupling coil array comprising one or more local coupling coils and being configured to:
be electrically connected to a second local coil device located above the examined portion of the subject to receive a second intermediate frequency signal from the second local coil device, wherein the second intermediate frequency signal is generated based on a second magnetic resonance signal received by the second local coil device from the subject; and
transmit the second intermediate frequency signal; and
a second receiving coupler comprising a second receiving coil array, the second receiving coil array comprising one or more second receiving coils and being located below the trajectory of the patient couch sliding over the magnetic resonance main body, and the second receiving coil array being configured to:
receive the second intermediate frequency signal from the local coupling coil array in response to the local coupling coil array at least partially overlapping with the second receiving coil array.

5. The wireless transmission device of claim 4, further comprising a locking structure configured such that:
when the patient couch slides to a locking position, the locking structure locks the second receiving coupler to the local coupler, and
when the patient couch slides to an unlocking position, the locking structure unlocks the second receiving coupler from the local coupler.

6. The wireless transmission device of claim 4,
wherein the second receiving coupler further comprises a second receiving auxiliary coil, wherein the second receiving coil array is arranged in the second receiving auxiliary coil, and the second receiving auxiliary coil is configured to transmit at least one of a second power signal, a second tuning control signal and a second local oscillator signal, and
wherein the local coupler further comprises a coupling auxiliary coil, wherein the local coupling coil array is arranged in the coupling auxiliary coil, and the local coupler is configured to perform:
the coupling auxiliary coil receiving at least one of the second power signal and the second tuning control signal from the second receiving auxiliary coil in response to the coupling auxiliary coil at least partially overlapping with the second receiving auxiliary coil; or
a component of the local coupler that is configured to transmit the second local oscillator signal receiving the second local oscillator signal from the second receiving auxiliary coil in response to the component of the local coupler that is configured to transmit the second local oscillator signal at least partially overlapping with the second receiving auxiliary coil, wherein the component of the local coupler that is configured to transmit the second local oscillator signal is the local coupling coil array or the coupling auxiliary coil.

7. A magnetic resonance imaging apparatus, comprising:
a magnetic resonance main body;
a patient couch;
a wireless transmission device of claim 4; and
a first local coil device comprising:
a first magnetic resonance coil configured to detect a first magnetic resonance signal emitted by a subject; and
a first frequency converter configured to convert the first magnetic resonance signal into a first intermediate frequency signal.

8. The magnetic resonance imaging apparatus of claim 7, further comprising a second local coil device comprising:
a second magnetic resonance coil configured to detect a second magnetic resonance signal emitted by the subject; and
a second frequency converter configured to:
convert the second magnetic resonance signal into a second intermediate frequency signal; and
transmit the second intermediate frequency signal to a local coupler in response to the second local coil device being electrically connected to the local coupler.

9. The magnetic resonance imaging apparatus of claim 7, wherein the first local coil device is a head coil device, a neck coil device, a head and neck coil device, a spine coil device, a chest coil device, a knee coil device, or an ankle coil device.

10. The magnetic resonance imaging apparatus of claim 7, wherein the second local coil device is a body coil device or a flexible coil device.
